(19) United States
(12) Patent Application Publication
(10) Pub. No.: US 2025/0073066 A1
(43) Pub. Date: Mar. 6, 2025

(54) INSTRUMENT STABILIZATION FOR MICROSURGERY

(71) Applicant: Alcon Inc., Fribourg (CH)

(72) Inventors: Patrick TERRY, Goleta, CA (US); Satish YALAMANCHILI, Irvine, CA (US); Russell FINLAY, Keller, TX (US); Nanhong LOU, Southlake, TX (US)

(21) Appl. No.: 18/816,969

(22) Filed: Aug. 27, 2024

Related U.S. Application Data
(60) Provisional application No. 63/579,938, filed on Aug. 31, 2023.

(51) Int. Cl. A61F 9/007; G16H 20/00

(52) U.S. Cl. CPC A61F 9/007; G16H 20/00

(57) ABSTRACT

A system for performing ophthalmic treatments includes a handpiece configured to be held by a hand of a surgeon. An end effector is mounted to the handpiece and configured to manipulate tissue of a patient, such tissues of the eye when performing an ophthalmic treatment. The system includes a motion sensor configured to sense movement of the handpiece and one or more stabilizing actuators configured to stabilize movement of the handpiece in response to movement of the hand of the surgeon. A controller is coupled to the motion sensor and the one or more stabilizing actuators and is configured to instruct the one or more stabilizing actuators to compensate for motion detected by the motion sensor. The controller may include a high-pass filer with the output of the high-pass filter used to control the stabilizing actuators. The parameters of the high pass filter may be adjusted throughout an ophthalmic treatment.

### CROSS-REFERENCE TO RELATED APPLICATIONS

This application claims priority to U.S. Provisional Application No. 63/579,938, filed on Aug. 31, 2023, which is hereby incorporated by reference in its entirety.

### BACKGROUND

The present disclosure relates generally to performing microsurgery, such as ophthalmic surgery.

Some surgical treatments require manipulation of very small and delicate structures. For example, ophthalmic surgical treatments with respect to the retina, vitreous, lens, trabecular meshwork, or other structures of the eye must be performed with small and precise movements in order to have the desired effect and avoid causing damage.

It would be an advancement in the art to facilitate the performance of microsurgery.

### SUMMARY

In one aspect of the invention, a system for performing ophthalmic treatments includes a handpiece configured to be held by a hand of a surgeon. An end effector is mounted to the handpiece and configured to manipulate tissue of a patient. The system includes a motion sensor configured to sense movement of the handpiece and one or more stabilizing actuators configured to stabilize movement of the handpiece in response to movement of the hand of the surgeon. A controller is coupled to the motion sensor and the one or more stabilizing actuators and is configured to instruct the one or more stabilizing actuators to compensate for motion detected by the motion sensor.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, and may admit to other equally effective embodiments.

FIG. 1A illustrates a surgical instrument with a remote stabilizing actuator in accordance with certain embodiments.

FIG. 1B illustrates a surgical instrument with stabilizing actuators affixed thereto in accordance with certain embodiments.

FIG. 2 illustrates a hand-worn device with stabilizing actuators in accordance with certain embodiments.

FIGS. 3A and 3B illustrate an instrument incorporating stabilizing actuators between a handpiece and a distal portion in accordance with certain embodiments.

FIG. 4 illustrates an instrument incorporating stabilizing actuators between a handpiece and fingers of a surgeon in accordance with certain embodiments.

FIG. 5 is a schematic diagram illustrating a system for controlling stabilizing actuators in accordance with certain embodiments.

### DETAILED DESCRIPTION

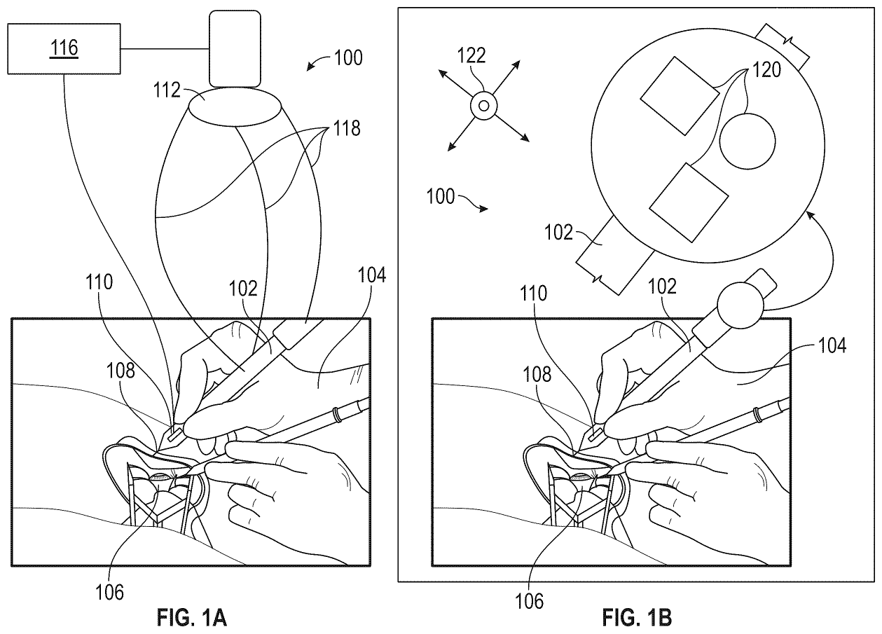

Referring to FIG. 1A, in some embodiments, a system 100 includes a handpiece 102 held by the hand 104 of a surgeon performing microsurgery, such as an ophthalmic treatment with respect to an eye 106 of a patient. An end effector 108 is mounted to the handpiece 102 and is used to manipulate tissues of the patient. For example, the end effector 108 may include forceps, a phaco-vit tool, an optical fiber for conducting light from a treatment laser, tube for supplying infusion fluid, an endoscope, or other type of surgical instrument. The ophthalmic treatment may include cataract surgery (e.g., phacoemulsification followed by placement of an intraocular lens (IOL)), glaucoma surgery (e.g., placement of an incision or shunt in the trabecular meshwork of the anterior chamber of the eye 106), vitrectomy, retinal attachment, retinal peeling, or other ophthalmic treatment.

A motion sensor 110 may be used to sense the position and orientation of one or both of the handpiece 102 and the end effector 108. The motion sensor 110 may be embodied as a device mounted to the handpiece 102 or end effector 108. The end effector 108 and handpiece 102 may be rigidly coupled to one another such that the position of one can be used to infer the position of the other. The motion sensor 110 may be embodied as a three-axis gyroscope sensing rotation about three mutually orthogonal axes. The motion sensor 110 may be embodied as a three-axis accelerometer sensing translation along three mutually orthogonal axes. The motion sensor 110 may incorporate both a three-axis gyroscope and a three-axis accelerometer.

Other types of sensors may also be used for the motion sensor 110. For example, one or more cameras may have the handpiece 102 in the field of view thereof. Images from the one or more cameras may then be processed to detect the position and orientation of the handpiece 102. In such embodiments, the handpiece 102 may have markings thereon to facilitate identification of the handpiece 102 as well as the orientation thereof in the images. In other embodiments, motion sensor 110 may include a local positioning system (LPS) using ultrasonic or radio frequency signals.

The handpiece 102 may be stabilized using stabilizing actuators to compensate for tremors, twitching or other involuntary movement of the hand 104 of the surgeon holding the handpiece 102. In the embodiment of FIG. 1A, the stabilizing actuators are implemented as cable actuators 112 remote from the handpiece 102 and connected to the handpiece 102 by one, two, three, or more cables 118. The cables 118 may be suspended at different positions from the cable actuators 112 such that the cables 118 connect to the handpiece 102 at different angles to enable translation of the handpiece 102 in three-dimensional space above the eye 106 of the patient. The cables 118 may be implemented as Bowden cables, i.e., a sheath with a cable sliding within the sheath, or may be simple cables extending from the cable actuators 112 to the handpiece 102 and that are tensioned to apply force to the handpiece 102. The cables 118 may be arranged to provide clearance for the surgeon to view the eye 106 of the patient, for a surgical microscope to be positioned over the eye 106 of the patient, or to provide clearance for other equipment. In addition to being used to provide stabilization, the cables 118 may also suspend the handpiece 102 such that it is approximately (e.g., at least 80% of the weight thereof) supported by the cables 118.

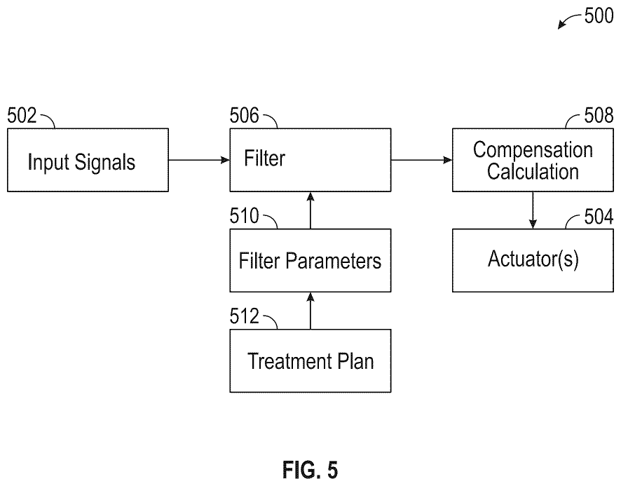

A controller 116 is coupled to the motion sensor 110 and to the cable actuators 112 by wired or wireless connections. The controller 116 controls the cable actuators 112 according to the outputs of the motion sensor 110 in order to reduce movement of the end effector 108 resulting from tremors, twitches, or other involuntary or excessively rapid movement of hand 104 of the surgeon. An example algorithm by which the controller 116 may do so is discussed below with respect to FIG. 5.

Referring to FIG. 1B, in an alternative embodiment of the system 100, the cable actuators 112 are replaced with one or more motors 120 mounted to the handpiece 102. The motors 120 may include three motors 120 having axes of rotation thereof aligned with three mutually orthogonal axis 122. The motors 120 include a spinning mass provided by the rotor thereof and/or by rotating masses that are spun by the motors 120. The controller 116 controls the supply of power to the motors 120 based on the output of the motion sensor 110. The controller 116 may accelerate and decelerate each motor 120 in order to induce a rotational moment on the handpiece 102 in order to reduce movement of the end effector 108 resulting from tremors, twitches, or other involuntary or excessively rapid movement of hand 104 of the surgeon as described in greater detail below with respect to FIG. 5. In some embodiments, the motors 120 may additionally or alternatively include linear actuators and masses actuated thereby, the linear actuators may be configured to induce movement of the one or more translating masses along some or all of the axes 122 or other mutually orthogonal axes.

The motors 120 may be located at (e.g., within 20 percent of the length of the handpiece 102 from) an end of the handpiece 102 opposite the end effector 108 as shown. Alternatively, the motors 120 may be placed at some other position, such as within a portion of the handpiece 102 grasped by the hand 104 of the surgeon. The motors 120 may be positioned such that the handpiece 102 is balanced, such as having a center of gravity at a point that is at a midpoint along the longest dimension of the handpiece 102, a point grasped by the fingers of the surgeon's hand 104, or other point on the handpiece 102.

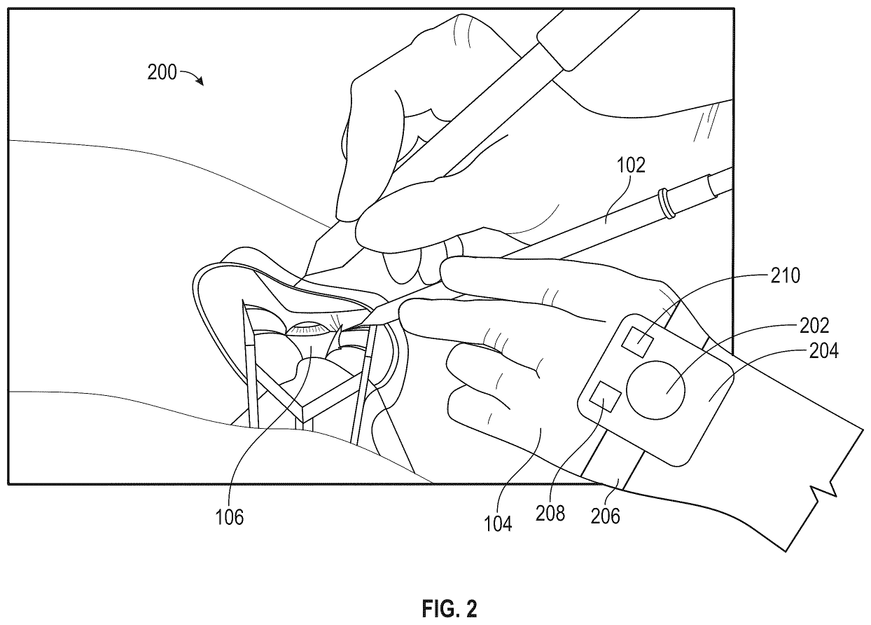

Referring to FIG. 2, in an alternative embodiment, a system 200 includes stabilizing actuators 202 mounted to the hand 104 of a surgeon. For example, a housing 204 having the stabilizing actuators 202 mounted thereto may be secured to a wrist band 206, glove, or other structure securing the housing 204 to the hand 104 of the surgeon.

The stabilizing actuators 202 may be implemented as a plurality of motors 120 (e.g., rotating motors and/or translating actuators as described above). The housing 204 may further have a motion sensor 208 mounted thereto and implemented using any of the approaches described above with respect to the motion sensor 110.

A controller 210 may be mounted to the housing 204 or may be remote from the housing and coupled by wires or wirelessly to the stabilizing actuators 202 and motion sensor 208. The controller 210 may control the stabilizing actuators 202 according to outputs of the motion sensor 208 as described below with respect to FIG. 5.

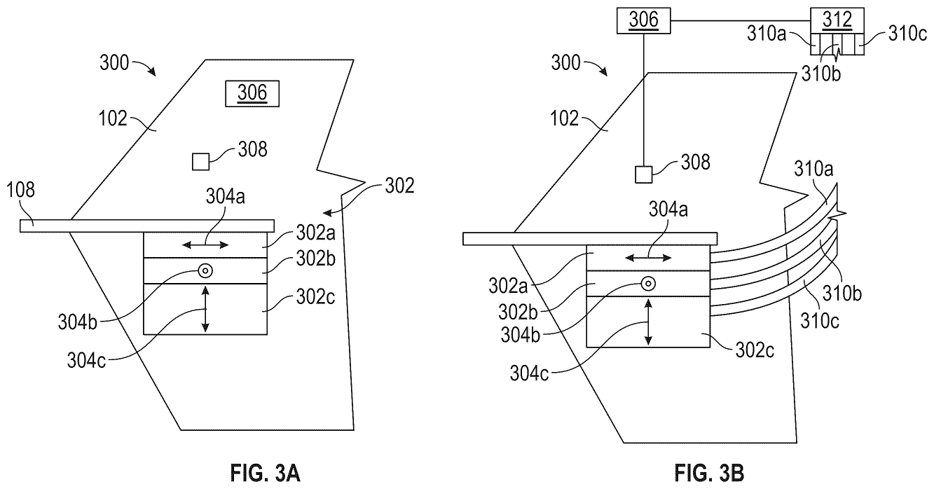

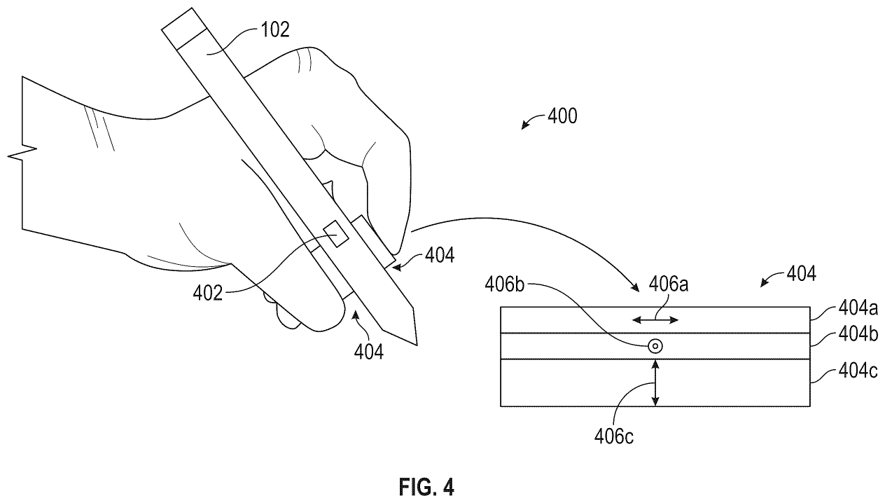

Referring to FIG. 3A, in some embodiments, a system 300 performs stabilization using stabilizing actuators 302 interposed between the handpiece 102 and the end effector 108. The system 300 may be used in combination with any of the other systems 100, 200, 300, 400 (see FIG. 4) described herein. The stabilizing actuators 302 may be controlled based on outputs of a motion sensor 308 sensing movement of the hand 104 of the surgeon. The motion sensor 308 may be implemented according to any of the approaches described above with respect to the motion sensor 110.

The stabilizing actuators 302 may include three to six stages 302a, 302b, 302, each stage of the stages 302a, 302b, 302c causing translation or rotation about one of three mutually orthogonal axes 304a, 304b, 304c. In the illustrated embodiment, the end effector 108 is mounted to and translated or rotated by a first stage 302a. The first stage 302a is mounted to and translated or rotated by a second stage 302b. The second stage 302b is mounted to and translated or rotated by a third stage 302c. Any number of stages may be stacked in this manner with the final stage, the third stage 302c in the illustrated example, being mounted to the handpiece 102.

The stages 302a, 302b, 302c may incorporate motors, linear actuators, or other actuators that control movement of the stages 302a, 302b, 302c according to signals from a controller 306. The controller 306 controls the stages 302a, 302b, 302c according to outputs of the motion sensor 308. The controller 306 may be mounted within the handpiece 102 or may be remote from the handpiece 102 and connected to the stages 302a, 302b, 302c and the motion sensor 308 by wires or wirelessly. The controller 306 may control the stages 302a, 302b, 302c according to outputs of the motion sensor 308 as described below with respect to FIG. 5.

As shown in FIG. 3B, the stages 302a, 302b, 302c may be actuated by force transmitted through lines 310a, 310b, 310c embodied as cables (bare or Bowden cables), tubes conducting pressurized air or fluid, or other type of lines for transmitting force. Force transmitted through the lines 310a, 310b, 310c may be provided by one more remote actuators 312, such as one or more cable actuators, one or more pneumatic pumps and valves, and/or one or more hydraulic pumps and valves. The supply of force by the remote actuators 312 may be controlled by the controller 306 according to outputs of the motion sensor 308. The controller 306 may be connected to the remote actuators by wires or wirelessly.

Referring to FIG. 4, in some embodiments a system 400 includes a handpiece 102 incorporating a motion sensor 402, such as a motion sensor according to any of the approaches described above with respect to the motion sensor 110. One or more stabilizing actuators 404 are interposed between the handpiece 102 and the hand 104 of the surgeon. For example, the one or more stabilizing actuators may be interposed between the thumb and forefinger of the surgeon and the handpiece 102. The stabilizing actuators 404 may be mounted to the handpiece 102 itself or be mounted to a glove worn on the hand 104 of the surgeon.

The one or more stabilizing actuators 404 may each include from three to six actuator stages 404a, 404b, 404c, each actuator stage 404a, 404b, 404c performing translation along or rotation about a corresponding axis 406a, 406b, 406c, the axes 406a, 406b, 406c being mutually orthogonal. In the illustrated embodiment, a surface that is rotated or translated by the first stage 404a is exposed and contacted by the hand 104 of the surgeon. The first stage 404a is mounted to and translated or rotated by a second stage 404b. The second stage 404b is mounted to and rotated or translated by a third stage 404c. The third stage 404c is mounted to the handpiece 102 in some embodiments. In other embodiments, the first stage 404a is mounted to a glove worn by the surgeon and the third stage 404c is placed in contact with the handpiece 102 during use. In still other embodiments, both the first stage 404a and third stage 404c are affixed to a globe and the handpiece 102, respectively. Any number of stages may be stacked as described above with a first stage contacting or mounted to the hand 104 of the surgeon and a final stage contacting or mounted to the handpiece 102. The actuator stages 404a, 404b, 404c may have some or all of the attributes of the stages 302a, 302b, 302c described above.

A controller is coupled to the motion sensor 402 and the actuator stages 404a, 404b, 404c. The controller controls the actuator stages 404a, 404b, 404c according to outputs of the motion sensor, such as according to the approach described below with respect to FIG. 5.

Referring to FIG. 5, a system 500 includes a motion sensor 502, such as a motion sensor 110, 208, 308, 402 according to any of the embodiments disclosed hereinabove. The system 500 further includes one or more stabilizing actuators 504, such as a cable actuator 112, motors 120, stabilizing actuators 202, stabilizing actuators 302, or stabilizing actuators 404 according to any of the foregoing embodiments.

The system 500 includes a high-pass filter 506. The high-pass filter 506 removes low-frequency motion information from signals received from the motion sensor 502. In particular, low-frequency motion, i.e., movement at frequencies below the cutoff frequency of the high-pass filter 506, will most likely correspond to deliberate and/or safe movements of the surgeon's hand 104 and therefore the low frequency motion is not subject to compensation by activating the stabilizing actuators 202. The high-pass filter 506 may filter each dimension of the output of the motion sensor 502 individually, i.e., each translational dimension and each rotational dimension. Alternatively, all dimensions may be filtered simultaneously. The output of the high-pass filter 506 may be input to a compensation calculating module 508, which calculates corresponding instructions to the stabilizing actuators 504. For example, movement (translational and/or rotational) represented in the output of the high-pass filter 506 may be translated into an equal and opposite movement. The equal and opposite movement may then be translated into instructions to the stabilizing actuators 504 to approximate the equal and opposite movement. The frequency at which the compensation calculating module 508 calculates the equal and opposite movement and transmits instructions to the stabilizing actuators 504 may be greater than the movement that is being compensated for, e.g., greater than the frequency response of the stabilizing actuators 504 in combination with the structures actuated thereby.

In some embodiments, the filter parameters 510 used to perform high-pass filtering by the high-pass filter 506 may be adjusted dynamically, such as the cutoff frequency, slope of the roll-off of the filter, order (first order, second order, third order, etc.), or other coefficients of the high-pass filter 506 that determine other aspects of operation of the high-pass filter 506. The filter parameters 510 may be adjusted dynamically by a surgeon. For example, one stage of an ophthalmic treatment may require greater precision than another. Accordingly, the cutoff frequency may be raised for a first portion requiring less precision and then lowered for a second portion requiring greater precision. In some embodiments, the filter parameters 510 for each portion of an ophthalmic treatment may be specified in a treatment plan 512. A surgeon may therefore change the filter parameters 510 by providing an input (key press, touch screen gesture, voice command, etc.) to the system 500 indicating a transition from one portion of the treatment plan to another.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

1. A system for performing ophthalmic treatments, the system comprising:
a handpiece configured to be held by a hand of a surgeon;
an end effector mounted to the handpiece and configured to manipulate tissue of a patient;
a motion sensor configured to sense movement of the handpiece;
one or more stabilizing actuators configured to stabilize movement of the handpiece in response to movement of the hand of the surgeon; and
a controller coupled to the motion sensor and the one or more stabilizing actuators, the controller configured to instruct the one or more stabilizing actuators to compensate for motion detected by the motion sensor.

2. The system of claim 1, wherein the one or more stabilizing actuators are mounted to the handpiece.

3. The system of claim 2, wherein the one or more stabilizing actuators are mounted to the handpiece between the end effector and the handpiece.

4. The system of claim 2, wherein the one or more stabilizing actuators are configured to be interposed between fingers of the hand of the surgeon and the handpiece.

5. The system of claim 2, wherein the one or more stabilizing actuators comprise a plurality of motors, each motor of the plurality of motors, each motor of the plurality of motors configured to spin about an axis of a plurality of mutually orthogonal axes.

6. The system of claim 2, wherein the one or more stabilizing actuators comprise a plurality of translating stages, each stage configured to translate along one axis of a plurality of mutually orthogonal axis.

7. The system of claim 2, wherein the one or more stabilizing actuators comprise a plurality of cable actuators mounted to the handpiece by a plurality of cables.

8. The system of claim 1, wherein the one or more stabilizing actuators are configured to mount to the hand of the surgeon.

9. The system of claim 1, wherein the motion sensor includes at least one of a three-axis accelerometer or a three-axis gyroscope.

10. The system of claim 1, wherein the controller comprises a high-pass filter, the controller configured to:
process outputs of the motion sensor using the high-pass filter to obtain filtered outputs; and
instruct the one or more stabilizing actuators to compensate for movement indicated in the filtered outputs.

11. The system of claim 10, wherein the controller is further configured to:
receive an input; and
in response to the input, adjust one or more parameters of the high-pass filter.

12. The system of claim 11, wherein the input is a treatment plan for an ophthalmic treatment.

13. The system of claim 11, wherein the one or more parameters include one or more of:
a cutoff frequency;
a roll-off slope; and
an order.

14. A method for performing ophthalmic treatments, the method comprising:
sensing, by a motion sensor, movement induced by a hand of a surgeon holding a handpiece having an end effector mounted thereto, the end effector being configured to manipulate tissue of a patient; and
instructing, by a controller coupled to the motion sensor, one or more stabilizing actuators to reduce an amount of the movement transferred to the tissue of the patient through the end effector.

15. The method of claim 14, wherein the one or more stabilizing actuators are mounted to the handpiece.

16. The method of claim 14, wherein the one or more stabilizing actuators are mounted to the handpiece between the end effector and the handpiece.

17. The method of claim 14, the one or more stabilizing actuators are configured to be interposed between fingers of the hand of the surgeon and the handpiece.

18. The method of claim 14, wherein the one or more stabilizing actuators are mounted to the hand of the surgeon.

19. The method of claim 14, further comprising:
processing outputs of the motion sensor using a high-pass filter to obtain filtered outputs; and
instructing, by the controller, the one or more stabilizing actuators according to the filtered outputs.

20. The method of claim 14, wherein the motion sensor includes at least one of a three-axis accelerometer or a three-axis gyroscope.
